(19) United States
(12) Patent Application Publication
(10) Pub. No.: US 2025/0090619 A1
(43) Pub. Date: Mar. 20, 2025

(54) SINGLE PROCESS FOR SEQUENTIAL EXTRACTION OF PRODUCTS FROM GREEN TEA LEAVES

(71) Applicant: Council of Scientific and Industrial Research, New Delhi (IN)

(72) Inventors: Mohit Sharma, Palampur Himachal Pradesh (IN); Mehak Sharma, Palampur Himachal Pradesh (IN); Vivesh Sood, Palampur Himachal Pradesh (IN)

(21) Appl. No.: 18/580,340

(22) PCT Filed: Jul. 15, 2022
(86) PCT No.: PCT/IN2022/050641

§ 371 Date: Jan. 18, 2024

(30) Foreign Application Priority Data
Jul. 19, 2021 (IN) 202111032421

(51) Int. Cl. A61K 36/82; A23F 3/22; A61K 31/353; A61K 31/522; B01D 11/02; B01D 15/08

(52) U.S. Cl. CPC A61K 36/82; A23F 3/22; A61K 31/353; A61K 31/522; B01D 11/0288; B01D 15/08; A61K 2236/33; A61K 2236/35; A61K 2236/37; A61K 2236/51; A61K 2236/53

(57) ABSTRACT

The present invention discloses an integrated, cost effective process for simultaneous isolation and purification of wax, chlorophyll, Catechin from fresh green tea (Camellia sinensis) leaves. Further, process for isolation and purification of caffeine from the residual tea leaves left after the extraction of above said products. Optimization of various process parameters has also been done. In addition to this the quality of products was also evaluated on the basis of TLC, FTIR, HPLC, GC-MS, method.

### FIELD OF THE INVENTION

The present invention relates to a single process for sequentially extracting products from green tea leaves. The invention discloses an integrated, cost effective process for simultaneous isolation and purification of wax, chlorophyll, catechin from fresh green tea (Camellia sinensis) leaves. Further, residual tea leaves can be processed for the isolation and purification of caffeine present in it.

### BACKGROUND OF THE INVENTION

Tea (Camellia sinensis) is an evergreen shrub which is native to East Asia. It is consumed worldwide and is second in popularity only to water. About three-thousand million kilograms of tea is produced and consumed yearly. The tea plant is used from ages for its bud and two or three leaves for the production of tea beverage but now with progress in science it is used for production of catechin. Earlier all the studies which were investigated for Camellia sinensis for the extraction of catechin are listed as follows. Fukuda et al., US patent number 2007/0082073A1 has focused on extraction of catechin and is not using the plant to the fullest. Okada et al., U.S. Pat. No. 4,248,789 has performed extraction at various polarities and the major focus is on only a single product i.e. catechin. Harsh Pratap Singh et al., WO2013/118152A1 had used the same plant material and had focused on bio amino acids. In this discovery a wide perspective is considered, i.e. the plant leaves can not only be used for the above two purposes but the usage of leaf can be extended for the development of other valued products too. Firstly, leaves are dipped in a solvent to extract lipid components of the same. This lipid layer can be used in cosmetic industry. Then the second extraction of the same leaves is done with a solvent to extract green pigment from plants. This pigment is of utmost importance in food industry/cosmetic industry/textile industry as a natural coloring agent. The present invention further relates to extraction of a brown colored compound known as catechin which is an antioxidant, anti-mutagenic, anti-bacterial, anti-viral and has protective effect from diseases like diabetes, cancer, cardiovascular disease and inflammation. In the course of extensive studies on the medicinal effects of tea components, it has been found that the tea catechin which are the major components of the so called tea tannin, possess strong physiological activity. As a result of further investigations, a novel method has been discovered which enables the production of catechin economically. The leaves left out (residual), after the extraction of catechin, are further processed for the extraction of caffeine which is considered as most widely consumed stimulant in the world and is naturally present in tea. Studies have linked consumption of caffeine with various health benefits. About percent of total production of caffeine is used in beverage and about 25 percent of total production is used in pharmaceutical preparations and rest for other purpose. This process is efficient and economic as the plant leaves are used to the fullest. Generally only the upper part of the plant has been used and the rest of the leaves remain unused but in this process lower leaves can also be used for production of different products like wax, chlorophyll, catechin and caffeine. Structures of these compounds are elucidated below:

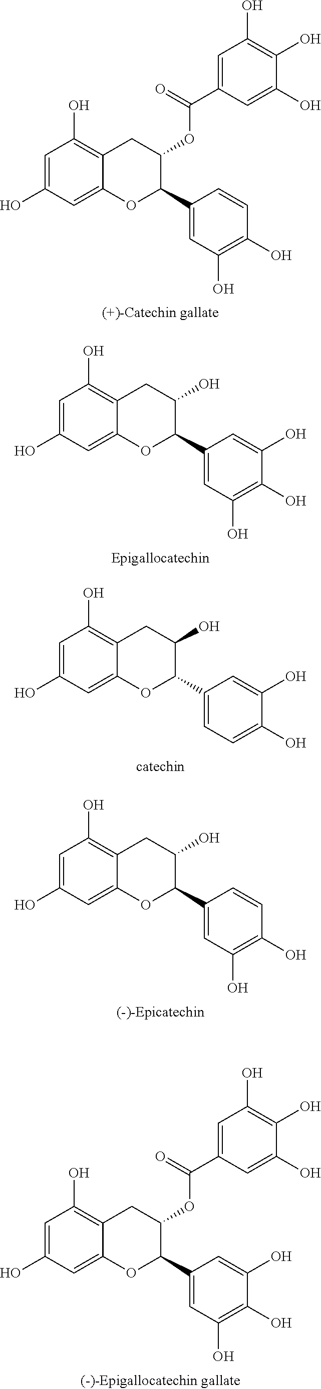 [SMILES: O=C(O[C@H]1Cc2c(O)cc(O)cc2O[C@@H]1c1ccc(O)c(O)c1)c1cc(O)c(O)c(O)c1 ; Oc1cc(O)c2c(c1)O[C@H](c1cc(O)c(O)c(O)c1)[C@@H](O)C2 ; Oc1cc(O)c2c(c1)O[C@@H](c1ccc(O)c(O)c1)[C@H](O)C2 ; Oc1cc(O)c2c(c1)O[C@H](c1ccc(O)c(O)c1)[C@@H](O)C2 ; O=C(O[C@H]1Cc2c(O)cc(O)cc2O[C@@H]1c1cc(O)c(O)c(O)c1)c1cc(O)c(O)c(O)c1]

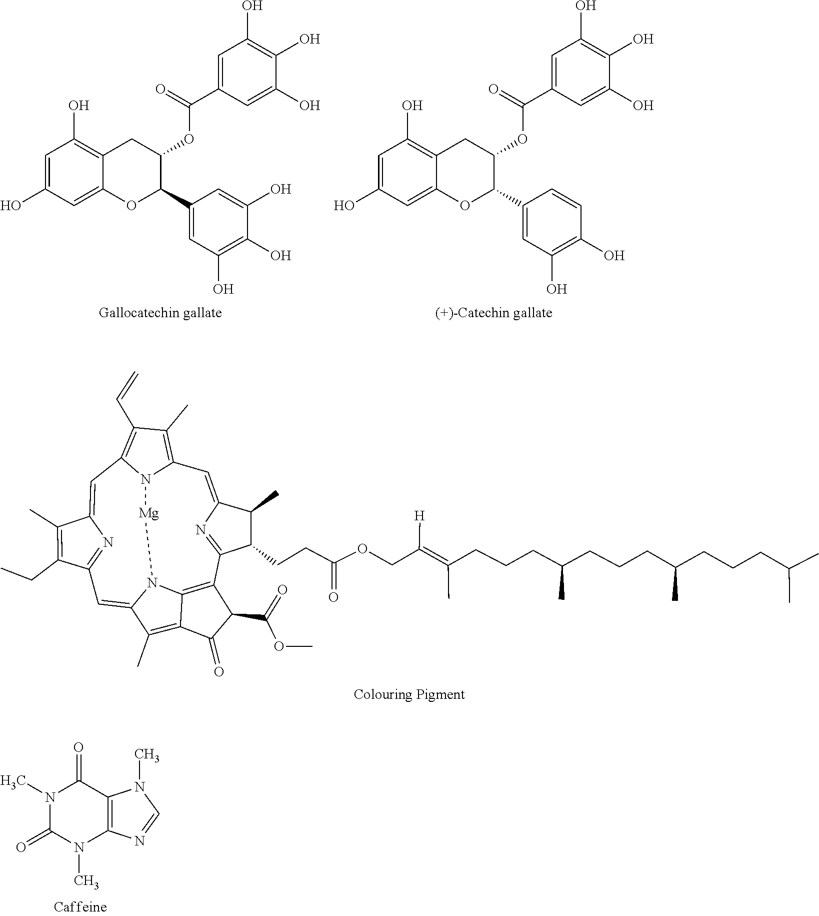

### SUMMARY OF THE INVENTION

Accordingly, the present invention provides a single process for sequential extraction of four products from green tea leaves. The products (wax, coloring pigment, catechin and caffeine) extracted are dipped in the alkane solvent such as hexane for 2-3 hours at room temperature to get a solution. Then filter the solution to get the residual leaves and filtrate. By drying the filtrate, the wax is produced. Dip the residual leaves in alcohol to get the green color solution and left-over leaves. Dry the green color solution to get the green color pigment i.e. chlorophyll. To obtain extract and residual spent leaves, hydro-extract the pigment for 30 minutes before drying the left-over leaves. Purify the extract by microfiltration by adsorbent resin-A and resin-C whereas resin-A is polystyrene. After elution of the extract a brown color, catechin powder is formed. The residual spent leaves are hydro-extracted with sodium carbonate at 100° C. for 1 hour. Through liquid-liquid extraction with the Dichloromethane, caffeine is recovered by cooling the extract. In the present invention both the upper and lower leaves of plant material has been used. The polystyrene adsorbent i.e. resin-A used in the process is of low cost. The invention shows that the usage of resin-A gives 3.59 g catechin whereas resin-C gives 1.32 g catechin. The process claims for extraction of cuticular lipid layer, coloring pigment, Catechin and caffeine from fresh green tea leaves stepwise. The residual/spent leaves left after extractions can be used as compost.

The main aspect of the invention related single process involves sequentially extracting products from green tea leaves comprising the steps:
a.) providing the green tea leaves, obtained from Camellia sinensis,
b.) dipping the green leaves in alkane solvent for 2-3 hours at room temperature to obtain a solution,
c.) filtering the solution as obtained in step (b) to obtain filtrate and residual leaves,
d.) drying the filtrate of step (c) to obtain wax as first product,
e.) dipping the residual leaves as obtained in step (c) in alcohol, followed by heating at 30-42° C. for 1-2 hours to obtain a green color solution & left over leaves,
f.) drying the green color solution as obtained in step (e) to obtain coloring pigment as second product.
g.) drying the left over leaves as obtained in step (e) followed by hydro extraction in ratio 1:10 at 60° C. for 30 mins to obtain extract & residual spent leaves,
h.) purifying the extract of step (g) by micro filtration 0.1-0.5-micron pore size,
i.) passing the purified extract through a adsorbent resin-A and resin-C,
j.) eluting the extract as obtained in step (h) with alcohol to obtain brown colored catechin powder as a third product,
k.) hydro extracting of residual spent leaves obtaining in step (g) with the sodium carbonate at 100° C. for 1 hour,
l.) cooling the extract as obtained in step (k) to recover caffeine as fourth product by the liquid-liquid extraction with the solvent.

In view of the process, the product obtained is selected from the group consisting of wax, coloring pigment, catechin & caffeine.

In one of the embodiments, the alkane solvent is hexane.

In one of the embodiments, the yield of the first product i.e. wax is 0.07%.

In yet another embodiments, the yield of the second product i.e. green coloring pigment is 8.6%.

In yet another embodiment both the upper and lower leaves of Camellia sinensis. is used for production of different products like wax, chlorophyll, catechin and caffeine.

Another embodiment involves resin-A as polystyrene resin.

Another aspect of the invention is related to catechin for use as an antioxidant, anti-mutagenic, anti-bacterial, anti-viral and has protective effect from diseases like diabetes, cancer, cardiovascular disease and inflammation.

Another embodiment of the invention involves solvent for further extraction which is Dichloromethane.

Further embodiments related to Dichloromethane containing caffeine was dried and purified by heating with minimum amount of alcohol to recover 0.07% caffeine.

In yet another embodiment, the yield of catechin from Resin-A is 3.59 g, resin-C give the yield of 1.32 g and yield of caffeine is 0.17 g.

### Objectives of the Invention

The main objective of the invention is to isolate four economically viable products from fresh green tea leaves using a single process viz., wax, coloring pigment, Catechin and caffeine.

### BRIEF DESCRIPTION OF THE FIGURE

FIG. 1 Flow chart showing the procedure of extraction of various products and comparison between the two resins used for extraction of Catechin from C. sinensis

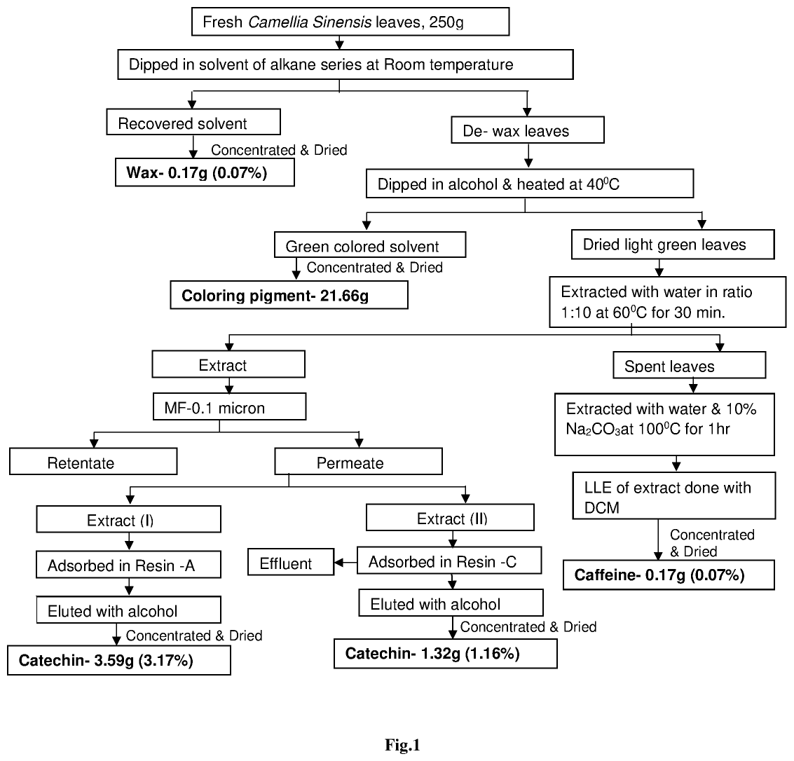

### DETAILED DESCRIPTION OF THE INVENTION

The leaf, stem, root, flower of Camellia sinensis were collected from Palampur in Himachal Pradesh (alt 1472 m) in November 2019. The plant material was submitted in the herbarium of CSIR-Institute of Himalayan Bioresource Technology, Palampur, Himachal Pradesh-176061, India and were identified with a voucher specimen (No. PLP18552).

The present invention provides an integrated process for the extraction of wax, chlorophyll, Catechin and caffeine from fresh green tea leaves step by step. In the first step, extraction of wax from the fresh green tea leaves is done by dipping the leaves in hexane for about 2-3 hours and then concentrating the hexane after filtration. Secondly, these leaves are dipped in alcohol and heated at 30-40° C. for 1-2 hours to extract green colored pigment from them. After removing the wax and chlorophyll, light green colored leaves are hydro extracted at 60° C. for 30 minutes with stirring the same. After extraction, extract has been filtered and then passed through selective polystyrene adsorbent resin to adsorb the Catechin in the same. Later, it has been eluted with alcohol and sprays dried to get the primary product which is brown colored Catechin powder. The leftover leaves (residual) are further processed by heating the same with water at 100° C. for 1 hour.

Later this water extract has been extracted with DCM to separate the caffeine from the same. Further DCM is dried to get the caffeine flakes. The isolated compounds may be elucidated by TLC, FTIR, NMR, and GC-MS.

The novelty in this process is extraction of four products namely wax, coloring pigment, Catechin and caffeine from the starting raw material i.e. fresh green tea leaves in stepwise manner simultaneously. Primarily, upper two leaves and bud are used for making quality tea products but remaining fresh tea leaves in the below whole plant remains unutilized. Therefore, the present invention is to develop a process which can yield valuable products by utilizing these remaining leaves through designing process steps which add value at each stage. Prior studies have reported extraction of products on dry processed material which adds drying cost to the process whereas the present invention is on value addition of fresh leaves. In addition to this, caffeine is produced by decaffeination of dry processed leaves using SFE which is capital intensive and operates at high pressure leading to extraction of caffeine only. But when caffeine is extracted by SFE result in sacrifice of some amount of Catechin also which makes this process less efficient in terms of extraction of Catechins. In addition to this, SFE has limitation of carbon dioxide through this technique. But the present process is simple, less costly and designed on fresh tea leaves. Tea is an evergreen plant and there is appreciable quantity of wax and coloring pigment which is underutilized, this invention is develop to extract these compounds which can be used in cosmetic and textile industry. Further, wax and color removed tea leaves result in better extraction rate and good yield of Catechin. The downstream purification step is also different and efficient done by using a special polystyrene based resin having more affinity for Catechin due to its specific small size (0.3-1.0 mm) chemistry and high surface areas compared to large size of other resins which makes former better adsorbent than the latter and not reported earlier. According to the rule, smaller the size of the beads more is the surface area and more separation and affinity towards the desired compound. The resin used in present invention is cheaper, efficient, economical and recyclable.

Moreover, the yield obtained through present invention is 3.61% (w/w on fresh basis) or 12.03% (w/w on dry basis) which is better and four times more than earlier reported 3.2% (w/w on dry basis) due to addition of sequential innovative steps. Lastly, a valuable pharmaceuticals and food compound caffeine extraction process is developed from spent leaves which are usually discarded.

### EXAMPLES

The following examples are given by way of illustration and therefore should not construed to limit the scope of the present invention.

### Example 1

100 g fresh green tea leaves were dipped in a solvent of alkane series for 2-3 hours at room temperature and filtrate is dried thereafter to recover 0.07 g (0.07%) wax. This wax is evaluated using GC-MS. GC-MS data was measured on QP2010 series (Shimadzu, Tokyo, Japan) fitted with AOC-20i auto sampler and FFAP1 column which results that the waxcontains esters, fatty acids, fatty alcohols, phospholipids and poly-phenols. Further leaves are dipped in alcohol and heated at 40° C. for 1-2 hours to get green colored solvent, which on drying yields 5.0 g (5.0%) coloring pigments. Later dried leaves are hydro-extracted at 60° C. for 30 minutes. This extract is further purified by 0.1 micron pore size microfiltration. Further this extract is passed through a selective polystyrene adsorbent resin (Resin-A), which on elution with alcohol yields 3.61 g (3.61%) catechin powder. Residual/spent leaves, after extraction of catechin, are further hydro-extracted along with sodium carbonate (10% of weight of residual leaves) at 100° C. for 1 hour. The extract was allowed to cool and then its liquid-liquid extraction was done with DCM to recover the caffeine from it. This DCM containing caffeine was dried and purified by heating with minimum amount of alcohol to recover 0.07 g (0.07%) caffeine.

### Example 2

250 g fresh green tea leaves were dipped in solvent of alkane series for 2-3 hours at room temperature and filtrate is dried thereafter to recover 0.15 g (0.06%) wax. Further leaves are dipped in alcohol at minus 15° C. for 1-2 hours to get green colored solvent, which on drying yields 5.88 g (2.35%) chlorophyll. Later dried leaves are hydro-extracted in the ratio of 1:10 at 60° C. for 30 minutes. This extract is further purified by 0.1 micron pore size microfiltration. Further this extract is divided into two parts of 1 liter each to study the adsorption capacity of the 2 different adsorbent resins. 1 liter of water extract was adsorbed in 100 ml of resin A (Polystyrene adsorbent resin) and another 1 liter extract was adsorbed in 100 ml of resin C for half an hour respectively which on elution with alcohol gives brown colored powder giving yield 2.52 g (2.23%) form Resin A and 2.18 g (1.92%) from resin C. Residual/spent leaves left after extraction of catechin are further hydro-extracted along with sodium carbonate (10% of weight of residual leaves) at 100° C. for 1 hour. The extract was allowed to cool and then its liquid-liquid extraction was done with DCM to recover the caffeine from it. This DCM containing caffeine was dried and purified by heating with minimum amount of alcohol to recover 0.17 g (0.07%) caffeine.

### Example 3

250 g fresh green tea leaves were dipped in solvent of alkane series for 2-3 hours at room temperature and filtrate is dried thereafter to recover 0.17 g (0.07%) wax. Further leaves are dipped in alcohol at room temperature for 1-2 hours to get green colored solvent, which on drying yields 8.13 g (3.25%) of coloring pigment. Later dried leaves are hydro-extracted in the ratio 1:10 at 60° C. for 30 minutes. This extract is further purified by 0.1 micron pore size microfiltration. Further this extract is divided into two parts of 1 liter each to study the adsorption capacity of the following resins. 1 liter of water extract was adsorbed in 100 ml of resin A (Polystyrene adsorbent resin) and another 1 liter extract was adsorbed in 100 ml of resin C for half an hour respectively which on elution with alcohol gives brown colored powder giving yield 2.28 g (2.01%) from Resin A and 1.97 g (1.74%) from resin C. Residual/spent leaves, after extraction of catechin, are further hydro-extracted along with sodium carbonate (10% of weight of residual leaves) at 100° C. for 1 hour. The extract was allowed to cool and then its liquid-liquid extraction was done with DCM to recover the caffeine from it. This DCM containing caffeine was dried and purified by heating with minimum amount of alcohol to recover 0.11 g (0.07%) caffeine. NMR of this caffeine (C1) and its standard (C2) has been done using NMR Bruker Avance™ 600 MHZ Spectrometer. The comparison of the ¹H and ¹³C NMR peaks was done for both the samples and it was found that the data matches thereby confirming it to be caffeine. The result of the ¹H NMR spectrum consist of the signals from three CH₃-groups at position 1,3,7 and one proton present at position number 8. The result of ¹³C NMR gave eight signals for the 5-Carbon atoms present at positions 2,4,5,6,8 of the ring and 3-carbon atoms of the methyl group attached to nitrogen of the ring present at 1,3,7 positions. The spectrum gave 4 signals for ¹H NMR and 8 for ¹³C NMR which are as follows:

| ¹H position | δ_{H} C1(Sample) | δ_{H} C2(standard) |
|---|---|---|
| 1 N—CH₃ | 3.408(s) | 3.388(s) |
| 3 N—CH₃ | 3.585(s) | 3.565(s) |
| 7 N—CH₃ | 3.990 | 3.976 |
| 8 | 7.504 | 7.493 |

| ¹³C Positions | δ_{C} C1(Sample) | δ_{C} C2(Standard) |
|---|---|---|
| 1 N—CH₃ | 28.083 | 28.037 |
| 2 | 151.907 | 151.847 |
| 3 N—CH₃ | 29.901 | 29.854 |
| 4 | 148.889 | 148.840 |
| 5 | 107.784 | 107.727 |
| 6 | 155.614 | 155.553 |
| 7 N—CH₃ | 33.737 | 33.696 |
| 8 | 141.535 | 141.506 |

These peaks in NMR spectrum predicted the structure of the compound as caffeine.

### Example 4

250 g fresh green tea leaves were dipped in solvent of alkane series for 2-3 hours at room temperature and filtrate is dried thereafter to recover 0.17 g (0.07%) wax. Further leaves are dipped in alcohol at room 40° C. for 1-2 hours to get green colored solvent, which on drying yields 21.66 g (8.6%) chlorophyll. This coloring pigment (Chlorophyll) is evaluated by FTIR spectroscopy using Shimadzu FTIR IR Prestige-21 IRAffinity-1 FTIR-8400S Shimadzu. The data of infrared transmittance was collected over a wave number ranged from 4000-500 cm⁻¹. The sample data was compared to reference to identify all the functional groups present in the sample. Major peaks of the sample are observed at 3340, 2970, 2931, 1750, 1650, 1465, 1379, 1128, 1107, 950 cm⁻¹. OH functional group provides peak at 3340.71 cm⁻¹ which is broad and this peak corresponds according to FTIR table which claims to give peak of OH at 3400-3700 cm⁻¹. The peaks in the region of 3000-2750 corresponds to alkanes i.e. CH₃ present in the compound. Further the carbonyl peaks are present at the wavenumber range of 1740 to 1800. The aromatic C═C bond gives beak in between 1600-1475 cm⁻¹, The carboxylic acid C—O gives peak at range of 1300-1000 cm⁻¹. The aliphatic amines give peaks at 1350-1000 cm⁻¹. These all ranges correspond to functional groups present in chlorophyll and when the peaks were matched to the reference the coloring pigment is found out to be chlorophyll which is a natural dye present in the plants. Later dried leaves are hydro-extracted in the ratio 1:10 at 60° C. for 30 minutes. This extract is further purified by 0.1 micron pore size microfiltration. Further this extract is divided into two parts of 1 liter each to study the adsorption capacity of the following resins. 1 liter of water extract was adsorbed in 100 ml of resin A (Polystyrene adsorbent resin) and another 1-liter extract was adsorbed in 100 ml of resin C for half an hour respectively which on elution with alcohol gives brown colored powder giving yield 3.59 g (3.17%) from Resin A and 1.32 g (1.16%) from resin C. Quality evaluation of catechin powder obtained from Resin-A and Resin-C has been done using Waters HPLC system equipped with 2707 auto sampler, 2998PDA detector with temperature control module, which confirms that the total catechin content in Resin-A powder is 53-57% while it is 45-48% in Resin-C powder.Residual/spent leaves, after extraction of catechin, are further hydro-extracted along with sodium carbonate (10% of weight of residual leaves) at 100° C. for 1 hour. The extract was allowed to cool and then its liquid-liquid extraction was done with DCM to recover the caffeine from it. This DCM containing caffeine was dried and purified by heating with minimum amount of alcohol to recover 0.17 g (0.07%) caffeine.

### Example 5

250 g fresh green tea leaves were dipped in solvent of alkane series for 2-3 hours at room temperature and filtrate is dried thereafter to recover 0.17 g (0.07%) wax. Further leaves are dipped in alcohol at room temperature of 40° C. for 1-2 hours to get green colored solvent, which on drying yields 21.66 g (8.6%) chlorophyll. Later dried leaves are hydro-extracted in the ratio 1:10 at 60° C. for 30 minutes. This extract is further purified by 0.1 micron pore size microfiltration. Further this extract is divided into two parts of 1 liter each to study the adsorption capacity of the following resins. 1 liter of water extract was adsorbed in 100 ml of resin A (Polystyrene adsorbent resin) and another 1-liter extract was adsorbed in 100 ml of resin C for half an hour respectively which on elution with alcohol gives brown colored powder giving yield 3.59 g (3.17%) from Resin A and 1.32 g (1.16%) from resin C. Quality evaluation of catechin powder obtained from Resin-A and Resin-C has been done using Waters HPLC system equipped with 2707 auto sampler, 2998PDA detector with temperature control module, which confirms that the total catechin content in Resin-A powder is 53-57% while it is 45-48% in Resin-C powder. Residual/spent leaves, after extraction of catechin, are further hydro-extracted along with sodium carbonate (10% of weight of residual leaves) at 100° C. for 1 hour. The extract was allowed to cool and then its liquid-liquid extraction was done with DCM to recover the caffeine from it. This DCM containing caffeine was dried and purified by heating with minimum amount of alcohol to recover 0.17 g (0.07%) caffeine.

### Example 6

Methods for simultaneous quantification of isolated compounds.

The quantitative analysis for wax was carried out by GC-MS. GC-MS data was measured on QP2010 series (Shimadzu, Tokyo, Japan) fitted with AOC-20i auto sampler and FFAP1 column. Helium (99.99% pure) was used as carrier gas with 4.8 ml/min total flow rate, linear velocity35.2 cm/see, pressure 69.3 kPa, split ratio 1.0 at a sampling rate of 1.0 scan/see, scan speed: 1666μ/s, interval 0.5 s. The samples diluted as 1 mg in 1 ml DCM (HPLC grade). The oven temperature was 120° C. Ion source temperature 200° C., interface temperature 250° C., injector temperature was maintained at 230° C. Its melting point is measured using m.p. measuring instrument from Barnstead Electro-thermal. Then further analysis of the green coloring pigment is done using Shimadzu FTIR IR Prestige-21 IRAffinity-1 FTIR-8400S Shimadzu. The quantitative analysis of Catechin was done using Waters HPLC system equipped with 2707 auto sampler, 2998PDA detector with temperature control module. A new synergie MAX RP C₁₂ column (4.6*250 mm) (Phenomenex) with particle size 4 micron was used. Stock solutions of all the standards were prepared individually in HPLC grade methanol at a concentration of 1.0 μg/μl. A gradient solvent system of acetonitrile (A) and water containing 0.01% trifluoroacetic acid (B) was used at constant flow rate of 1.0 ml/min. The gradient conditions were as follows: 0 min. 10% A, 3 min. 15% A, 5 min 20%, 8 min. 25% A and 10 min. 30% A. The gradient was again brought to 10% of solvent A upto 20 min. The injection volume was kept at 10 μl. The chromatogram spectrum was recorded at 200-700 nm by wavelength scan program and the final data was collected at 205 nm for each sample and standard. Data interpretation was carried out using Empower 2 software. Further the caffeine extracted from residual/spent tea leaves is quantified using the NMR Bruker Avance™ 600 MHz Spectrometer and same RP-HPLC method as mentioned above.

| | | |
|---|---|---|
| Properties | Resin A | Resin C |

| | | | | |
|---|---|---|---|---|
| Particle size | 0.3-1.0 | mm | 0.25-1.6 | mm |
| Specific surface area | 750 | m²/gm | 590 | m²/gm |

| | | |
|---|---|---|
| Moisture Content | 53-58% | 55-65% |
| Swelling Ratio | 1:1.42 | 1:1.24 |

| | | | | |
|---|---|---|---|---|
| (in ethanol) | | | | |
| Operating Temperature | 150° | C. | 130° | C. |

### Advantages of the Present Invention

1. This study provides an integrated stepwise method which enables producing four valuable/commercially useful products from same plant material.
2. It provides a process for extraction of wax from fresh green tea leaves which can be used in cosmetics.
3. It provides a process in which a green colored compound is extracted which on further conversion is soluble in oil and can be used as a colorant in industries.
4. This study provides a method which is cost effective and is increasing the yield of primary compound i.e. catechin.
5. This method provides catechin (an antioxidant, anti-mutagenic, anti-bacterial, anti-viral and has protective effect from diseases like diabetes, cancer, cardiovascular disease and inflammation) in powdered form which can be formulated as capsules/tablets for oral consumption.
6. It provides a method which can be used for formation of green tea extract.
7. It provides a process in which the residual/spent leaves can be used to extract caffeine.

### REFERENCES

1. Alberto Van Olphen, Herman Friedman, Neil Shaw, Nicholas Burdash and James Roger, Florida, 2007. Catechin Adjuvants.
2. Laurent Bazinet et al., 2009. Method for selectively and sequentially extracting catechins from green tea leaf.
3. Hitoshi Sato, Hideaki Ueoka, Keiji Shibata, Tokyo, 2010. Tea Extract.
4. Kenji Saito, Hiratsuka-shi, Tokyo, 2010. Method for producing low caffeine-tea extract.
5. Masahiro Fukuda, Hirokaz Takahahashi, Atsushi Konishi, Tokyo, 2011.Process for producing tea extract containing non-polymeric catechin.
6. Harsh Pratap Singh and Ajay Rana, CSIR-IHBT India 2013.A solvent free process for purification of free bio amino acid.
7. Quan vuong and Paul Daniel Roch, Australia, 2014. Article in separation and purification reviews. Caffeine in green tea.
8. Dias Indrasti, Nuri and Arwulan, Eko Hari Purnomo, and Nur Wulandari, Indonesia, 2018. Stability of chlorophyll as natural colorant: A review for suji leaves.
9. Nakajima, Makoto Kawasaki-shi, London, 2019. Beverage containing catechins and RebD and/or RebM.

The invention claimed is:

1-11. (canceled)

12. An integrated process for sequentially extracting products from green tea leaves, the integrated process comprising:
(a) providing green tea leaves obtained from Camellia sinensis;
(b) dipping the green tea leaves in an alkane solvent for 2 hours to 3 hours at room temperature to obtain a solution;
(c) filtering the solution of (b) to obtain a filtrate and residual leaves;
(d) drying the filtrate of (c) to obtain a wax as a first product;
(e) dipping the residual leaves of (c) in alcohol followed by heating at 30° C. to 42° C. for 1 hour to 2 hours to obtain a green-colored solution and leftover leaves;
(f) drying the green-colored solution of (e) to obtain a coloring pigment as a second product;
(g) drying the leftover leaves of (e) followed by hydroextraction in a ratio 1:10 at 60° C. for 30 minutes to obtain a first extract and residual spent leaves;
(h) purifying the first extract of (g) by micro filtration with a 0.1-micron to 0.5-micron pore size to obtain a purified first extract;
(i) passing the purified first extract of (h) through a polystyrene adsorbent Resin-A or through a Resin-C;
(j) eluting the purified first extract of (h) with alcohol to obtain a brown-colored catechin powder as a third product;
(k) hydroextracting the residual spent leaves of (g) with sodium carbonate at 100° C. for 1 hour to obtain a second extract; and
(l) cooling the second extract of (k) and recovering caffeine as a fourth product by liquid-liquid extraction of the second extract with dichloromethane solvent.

13. The integrated process of claim 12, wherein the first product is obtained with a yield of 0.07%.

14. The integrated process of claim 12, wherein second product is obtained with a yield of 8.6%.

15. The integrated process of claim 12, wherein the third product is catechin.

16. The integrated process of claim 12, wherein, in (i), the first extract is passed through the polystyrene adsorbent Resin-A and a yield of catechin from the polystyrene adsorbent Resin-A is 3.17%.

17. The integrated process of claim 12, wherein, in (i), the first extract is passed through the Resin-C and a yield of catechin from the Resin-C is 1.16%.

18. The integrated process of claim 12, wherein the caffeine is obtained with a yield of 0.07%.

19. The integrated process of claim 12, wherein alkane solvent is hexane.

20. The integrated process of claim 12, wherein the green tea leaves comprise both upper leaves and lower leaves of the Camellia sinensis.
